(19) United States
(12) Patent Application Publication
(10) Pub. No.: US 2023/0298766 A1
(43) Pub. Date: Sep. 21, 2023

(54) MOBILE TERMINAL, PROXIMITY DETECTION SYSTEM, METHOD, AND NON-TRANSITORY COMPUTER READABLE MEDIUM

(71) Applicant: NEC Corporation, Minato-ku, Tokyo (JP)

(72) Inventors: Akiko Suzuki, Tokyo (JP); Junichi Funada, Tokyo (JP)

(73) Assignee: NEC Corporation, Minato-ku, Tokyo (JP)

(21) Appl. No.: 18/017,472

(22) PCT Filed: Jul. 28, 2020
(86) PCT No.: PCT/JP2020/028864

§ 371 Date: Jan. 23, 2023

(51) Int. Cl. G16H 50/80; H04W 64/00

(52) U.S. Cl. CPC G16H 50/80; H04W 64/003

(57) ABSTRACT

A mobile terminal capable of grasping the possibility that a person had been in proximity to an infected person while taking his/her privacy into consideration is provided. A mobile terminal according to the present disclosure includes an extraction unit configured to extract, from an image of an identification code generated based on identification information assigned to a place and displayed at the place, the identification information, and store the extracted identification information in a storage device, an acquisition unit configured to acquire identification information stored in a storage device of a mobile terminal of an infected person, and a determination unit configured to determine whether or not the acquired identification information matches the identification information stored in the storage device of a user's own mobile terminal. The determination unit notifies the user of information about proximity of the user to the infected person based on a result of the determination.

### TECHNICAL FIELD

The present invention relates to a mobile terminal, a proximity detection system, a method, and a non-transitory computer readable medium.

### BACKGROUND ART

As a measure for preventing the spread of an infectious disease, it is desired to identify, for example, people who had been in proximity to a person who has been diagnosed as being infected with the infectious disease (hereinafter also referred to as an infected person) in a store or the like.

Patent Literature 1 discloses a technology for collecting information representing histories of the behaviors of users of a store based on a store ID for identifying the store and user IDs assigned to the users of the store, both of which are distributed (i.e., assigned) in each of a plurality of stores.

### CITATION LIST

### Patent Literature

Patent Literature 1: Japanese Unexamined Patent Application Publication No. 2018-169686

### SUMMARY OF INVENTION

### Technical Problem

However, in Patent Literature 1, individuals may be identified because the histories of their behaviors are recorded. Therefore, some people hesitate to use the system because of concern about their privacy or the like, and thus it is impossible to grasp (i.e., to know) the possibility that they had been in proximity to an infected person. Therefore, there has been a problem that it is not easy to grasp the possibility that a person had been in proximity to an infected person while having to take their privacy into consideration.

An object of the present disclosure is to solve the above-described problem and to provide a mobile terminal, a proximity detection system, a method, and a non-transitory computer readable medium capable of grasping the possibility that a person had been in proximity to an infected person while taking his/her privacy into consideration.

### Solution to Problem

A non-transitory computer readable medium according to the present disclosure stores a program for causing a computer to: extract, from an image of an identification code generated based on identification information assigned to a place and displayed at the place, the identification information, and store the extracted identification information in a storage device; acquire identification information stored in a storage device of a mobile terminal of an infected person; determine whether or not the acquired identification information matches the identification information stored in the storage device of a user's own mobile terminal; and notify the user of information about proximity of the user to the infected person based on a result of the determination.

A mobile terminal according to the present disclosure includes: an extraction unit configured to extract, from an image of an identification code generated based on identification information assigned to a place and displayed at the place, the identification information, and store the extracted identification information in a storage device; an acquisition unit configured to acquire identification information stored in a storage device of a mobile terminal of an infected person; and a determination unit configured to determine whether or not the acquired identification information matches the identification information stored in the storage device of a user's own mobile terminal, in which the determination unit notifies the user of information about proximity of the user to the infected person based on a result of the determination.

A proximity detection system according to the present disclosure includes: the above-described mobile terminal; and display means for generating an identification code based on identification information assigned to a place, and displaying the generated identification code.

A method according to the present disclosure includes: extracting, by a mobile terminal, from an image of an identification code generated based on identification information assigned to a place and displayed at the place, the identification information, and storing the extracted identification information in a storage device; acquiring, by the mobile terminal, identification information stored in a storage device of a mobile terminal of an infected person; determining, by the mobile terminal, whether or not the acquired identification information matches the identification information stored in the storage device of a user's own mobile terminal; and notifying, by the mobile terminal, the user of information about proximity of the user to the infected person based on a result of the determination.

### Advantageous Effects of Invention

According to the present disclosure, it is possible to provide a mobile terminal, a proximity detection system, a method, and a non-transitory computer readable medium capable of grasping the possibility that a person had been in proximity to an infected person while taking his/her privacy into consideration.

### BRIEF DESCRIPTION OF DRAWINGS

FIG. 1 is a block diagram showing a configuration of a mobile terminal according to a first example embodiment;

FIG. 2 is a block diagram showing a configuration of a proximity detection system according to a second example embodiment;

FIG. 3 is a schematic diagram showing an identification code according to the second example embodiment;

FIG. 4 is a schematic diagram showing an operation for acquiring a place performed in the proximity detection system according to the second example embodiment;

FIG. 5 is a flow chart showing an operation for creating an identification code and displaying the generated identification code on display means performed in a management server according to the second example embodiment;

FIG. 6 is a flowchart showing an operation for displaying an identification code performed in display means according to the second example embodiment;

FIG. 7 is a flowchart showing an operation for storing an identification code performed in mobile terminal according to the second example embodiment;

FIG. 8 is a schematic diagram showing an operation for identifying a proximity person and for notification thereof performed in the proximity detection system according to the second example embodiment;

FIG. 9 is a flowchart showing an operation for transmitting identification information to the management server performed in the mobile terminal according to the second example embodiment;

FIG. 10 is a flowchart showing an operation for transmitting identification information of an infected person to the mobile terminal performed in the management server according to the second example embodiment;

FIG. 11 is a flowchart showing an operation for notifying a user about information about proximity to an infected person performed in the mobile terminal according to the second example embodiment; and

FIG. 12 is a block diagram for showing an example of a hardware configuration of a computer.

### EXAMPLE EMBODIMENT

A specific example embodiment to which the present disclosure is applied will be described hereinafter in detail with reference to the drawings. In the drawings, the same reference numerals (or symbols) are assigned to the same components, and redundant descriptions thereof are omitted as appropriate for clarifying the description.

### First Example Embodiment

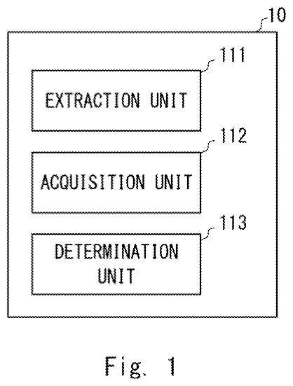

Firstly, a configuration of a mobile terminal according to a first example embodiment will be described with reference to FIG. 1. FIG. 1 is a block diagram showing a configuration of a mobile terminal 10 according to the first example embodiment. The mobile terminal 10 includes an extraction unit 111, an acquisition unit 112, and a determination unit 113.

The extraction unit 111 extracts identification information from an image of an identification code. The identification code is generated based on identification information assigned to a place and displayed at that place. The place where an identification code is assigned is a place used by users, such as a store and a facility.

The acquisition unit 112 acquires identification information extracted by a mobile terminal of an infected person.

The determination unit 113 determines whether or not the identification information of the infected person matches the identification information extracted by the user's own mobile terminal.

Next, operations performed by the mobile terminal 10 according to the first example embodiment will be described.

Firstly, the user of the mobile terminal 10 visits a place such as a store where an identification code is displayed. Then, according to the user's operation, the extraction unit 111 of the mobile terminal 10 extracts, from an image of an identification code generated based on identification information assigned to a place and displayed at the place, the identification information, and stores the extracted identification information in a storage device.

After that, the acquisition unit 112 acquires identification information stored in the storage device of the mobile terminal of an infected person. Next, the determination unit 113 determines whether or not the acquired identification information matches the identification information stored in the storage device of the user's own mobile terminal. Next, the determination unit 113 notifies the user of information about proximity of the user to the infected person based on the result of the determination.

The mobile terminal 10 according to the first example embodiment can notify the user that he/she might have been in proximity to an infected person by determining whether or not the identification information stored in the user's own mobile terminal matches the identification information of the mobile terminal 10 owned by the infected person. It should be noted that it is unnecessary to register any personal information such as an email address, and the user can be notified that he/she might have been in proximity to an infected person based on the identification information.

Therefore, in the mobile terminal 10, even people who hesitate to use the system because of concern about their privacy or the like can use the system and grasp (i.e., know) the possibility that they had been in proximity to an infected person. That is, in the mobile terminal 10, it is possible to easily grasp the possibility that the user had been in proximity to an infected person while his/her privacy is taken into consideration.

### Second Example Embodiment

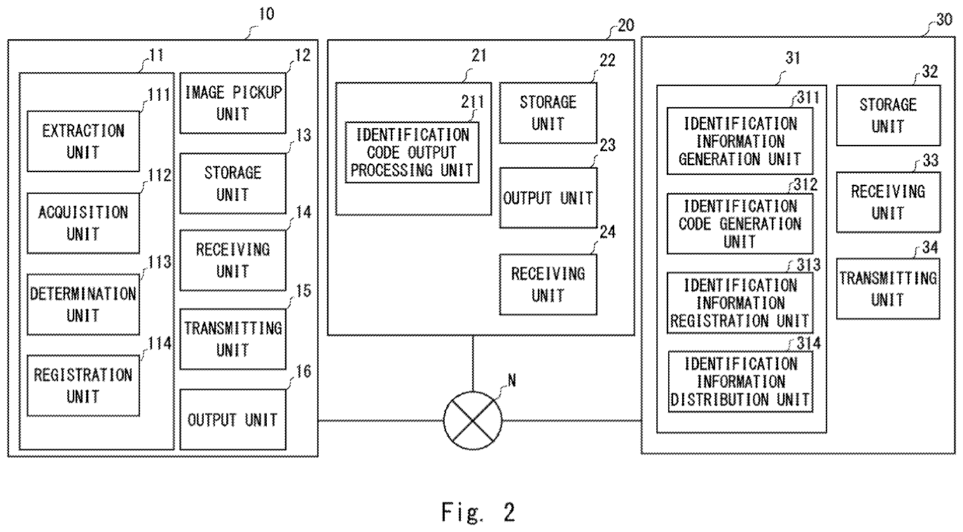

Firstly, a configuration of a proximity detection system 2 according to a second example embodiment will be described with reference to FIG. 2. FIG. 2 is a block diagram showing a configuration of the proximity detection system 2 according to the second example embodiment. The proximity detection system 2 includes a mobile terminal 10, display means 20, and a management server 30.

The mobile terminal 10 is a mobile-communication mobile terminal, such as a smartphone or a tablet-type computer, used by a user. The mobile terminal wirelessly communicates with the management server 30 through a network N such as the Internet.

The display means 20 is an arbitrary computer having a displaying function. The computer may be a portable computer such as a smartphone or a tablet-type computer, or a stationary computer such as a PC (Personal Computer) with a display device connected thereto. Display means 20 is installed in each of various places such as shops, facilities where a number of people visit, and event venues where a number of people gather. The display means 20 (i.e., each display means 20) communicates with the management server 30 through the network N wirelessly or through a cable.

The management server 30 is a server that manages the display means 20 and the proximity detection system 2.

Firstly, an outline of the proximity detection system 2 according to the second example embodiment will be described.

The management server 30 generates (encodes) an identification code based on identification information (such as text information) for identifying a combination of the date/time and the place where the display means 20 is installed. The display means 20 displays the identification code generated by the management server 30.

The user of the mobile terminal 10 visits a store or the like where the display means 20 is installed. The user operates the mobile terminal 10 and thereby makes (i.e., instructs) a camera or the like provided in the mobile terminal 10 takes an image of the identification code displayed by the display means 20. The mobile terminal 10 decodes the taken identification code and thereby extracts identification information, and stores the extracted identification information. By doing so, for all the places the user of the mobile terminal 10 has visited, identification information (i.e., a plurality of pieces of identification information) each of which corresponds to a combination of a respective one of the places and the date/time at which the user visited that place are accumulated in the mobile terminal 10.

When the user of the mobile terminal 10 (e.g., one of mobile terminals 10) is diagnosed as an infected person, i.e., as being infected with an infectious disease (e.g., as being infected with an infectious disease of interest), the mobile terminal 10 transmits the identification information (i.e., a plurality of pieces of identification information) stored in the user's own mobile terminal (i.e., in that mobile terminal itself) to the management server 30 according to an instruction from the user. The management server 30 receives the identification information from the mobile terminal 10. The management server 30 stores the identification information received from mobile terminal 10 as identification information of an infected person.

The management server 30 transmits the stored identification information of an infected person(s) to the mobile terminal 10 at predetermined intervals. The mobile terminal 10 receives the identification information of the infected person(s) from the management server 30, and determines whether or not the identification information stored in the user's own mobile terminal matches the identification information of the infected person. When they match each other, the mobile terminal 10 notifies the user of the mobile terminal 10 that he/she might have been in proximity to an infected person.

Note that a plurality of pieces of identification information may be stored in the mobile terminal 10. When a plurality of pieces of identification information are stored in the mobile terminal 10 of the infected person, the plurality of these pieces of identification information are transmitted to mobile terminals 10 of other users. Upon receiving the plurality of pieces of identification information, the mobile terminal 10 (i.e., each of the other mobile terminals 10) compares each of the plurality of received pieces of identification information with each of a plurality of pieces of identification information stored in the user's own mobile terminal. Then, when at least one of the plurality of received pieces of identification information matches at least one of the plurality of pieces of identification information stored in the user's own mobile terminal, the mobile terminal 10 (i.e., the user's own mobile terminal) notifies the user thereof that he/she might have been in proximity to an infected person.

### <Mobile Terminal 10>

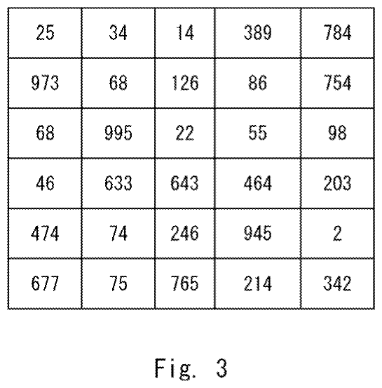

Next, a detailed configuration of the mobile terminal 10 according to the second example embodiment will be described with reference to FIG. 3. The mobile terminal 10 includes a control unit 11, an image pickup unit 12, a storage unit 13, a receiving unit 14, a transmitting unit 15, and an output unit 16.

The control unit 11 connects to the image pickup unit 12, the storage unit 13, the receiving unit 14, the transmitting unit 15, and the output unit 16, and controls each of the units that the control unit 11 has connected.

The image pickup unit 12 includes an image-pickup device such as a camera. The image pickup unit 12 supplies a taken image to the control unit 11. Note that the image pickup unit 12 may store the taken image in the storage unit 13.

The storage unit 13 includes, for example, a storage device such as an HDD (Hard Disk Drive), an SSD (Solid State Drive), or a flash memory. The storage unit 13 stores images taken by the image pickup unit 12. Further, the storage unit 13 stores identification information. The storage unit 13 may be set so as to retain identification information for a predetermined period such as 14 days.

The receiving unit 14 and the transmitting unit 15 connect to the network N, which may be the Internet, and perform wireless communication therethrough. The receiving unit 14 receives information from the management server 30 through the network N. The transmitting unit 15 transmits information to the management server 30 through the network N.

The output unit 16 includes a display device such as an LCD (Liquid Crystal Device) or an organic EL (Electro Luminescence) device, and visually conveys information supplied from the control unit 11 to the user. Further, the output unit 16 may include a sound output device such as a speaker, and auditorily conveys information supplied from the control unit 11 to the user.

The control unit 11 includes an extraction unit 111, an acquisition unit 112, and a determination unit 113.

The extraction unit 111 acquires a taken image (e.g., a photograph image) from the image pickup unit 12 and extracts (decodes) identification information from an identification code included in the taken image. The extraction unit 111 stores the extracted identification information in the storage unit 13.

The acquisition unit 112 acquires identification information of an infected person(s) from the management server 30 through the receiving unit 14. The acquisition unit 112 may acquire a plurality pieces of identification information of infected persons.

The determination unit 113 determines whether or not the identification information stored in the storage unit 13 matches the identification information of the infected person acquired by the acquisition unit 112. The determination unit 113 notifies the user of the mobile terminal 10 of information about his/her proximity to the infected person based on the result of the determination. When the identification information stored in the storage unit 13 of the user's own mobile terminal matches the identification information of the infected person, the determination unit 113 notifies, through the output unit 16, the user of the mobile terminal 10 that he/she might have been in proximity to the infected person.

A registration unit 114 acquires identification information from the storage unit 13. The registration unit 114 transmits the acquired identification information to the management server 30 through the transmitting unit 15.

### <Display Means 20>

Next, a detailed configuration of the display means 20 according to the second example embodiment will be described. The display means 20 includes a control unit 21, a storage unit 22, an output unit 23, and a receiving unit 24.

The control unit 21 connects to the storage unit 22, the output unit 23, and the receiving unit 24, and controls each of the units that the control unit 21 has connected. The control unit 21 includes an identification code output processing unit 211.

The identification code output processing unit 211 makes (i.e., instructs) the output unit 23 output the identification code that the receiving unit 24 has received from the management server 30. The identification code output processing unit 211 stores the received identification code received in the storage unit 22.

The storage unit 22 includes a storage device such as an HDD, an SSD, or a flash memory. The storage unit 22 stores, for example, an identification code(s) received from the management server 30 of the display means 20.

The output unit 23 includes a display device such as an LCD or an organic EL device, and visually conveys the identification code supplied from the control unit 21 to the user.

The receiving unit 24 connects to the network N, which may be the Internet, and performs wired or wireless communication. The receiving unit 24 receives information from the management server 30 through the network N.

### <Management Server 30>

Next, a detailed configuration of the management server 30 according to the second example embodiment will be described. The management server 30 includes a control unit 31, a storage unit 32, a receiving unit 33, and a transmitting unit 34.

The control unit 31 connects to the storage unit 32, the receiving unit 33, and the transmitting unit 34, and controls each of the units that the control unit 31 has connected. The control unit 31 includes an identification information generation unit 311, an identification code generation unit 312, an identification information registration unit 313, and an identification information distribution unit 314.

The identification information generation unit 311 generates identification information. The identification information is expressed by a string of numbers and/or a string of characters (e.g., alphabets). The identification information is unique to each combination of a date/time and a place of the display means 20. In other words, even for the same display means 20, the identification information changes according to the data/time. Similarly, even at the same date/time, the identification information differs from one display means 20 to another. For example, the identification information generation unit 311 randomly generates strings of numbers and/or strings of characters in such a manner that each of them is unique (i.e., they differ from one another). The place is identified, for example, based on place information. The time period indicates a predetermined time period, and is defined, for example, as 0:00 to 24:00.

The identification information generation unit 311 generates identification information at predetermined intervals. Then, the identification code generation unit 312 generates an identification code again (i.e., repeatedly) based on the identification information generated by the identification information generation unit 311. By doing so, it is possible to repeatedly update the identification information. Therefore, the assigned date/time corresponds to the predetermined period (i.e., the predetermined interval) at which the identification information is updated.

The identification code generation unit 312 generates an identification code based on the identification information. The identification code is, for example, a two-dimensional code such as a code to which a random number is assigned (hereinafter also referred to as a random-number code) or a QR code (Registered Trademark). Then, the identification code generation unit 312 transmits the generated identification code to the display means 20.

For example, the identification code generation unit 312 generates an identification code by encoding identification information by a method described below. FIG. 3 is a schematic diagram showing an identification code according to the second example embodiment. In the example shown in FIG. 3, the identification code generation unit 312 generates a plurality of random numbers and generates partial images corresponding to the generated random numbers. Then, the identification code generation unit 312 expresses the identification information by disposing the generated partial images at predetermined positions in a grid pattern, and thereby generates a random-number code.

Note that the identification code generation unit 312 may generate partial images to which figures (or diagrams) such as dots corresponding to random numbers are assigned, and express the identification information by arranging them in a predetermined pattern.

Note that it is sufficient if the identification information is unique at least to the display means 20, and does not necessarily have to be updated at predetermined intervals such as every day or every hour. In this case, the identification information generation unit 311 does not have to generate again (i.e., repeatedly) the identification information at predetermined intervals.

The identification information registration unit 313 stores the identification information received from the mobile terminal 10 of the user who has been diagnosed as an infected person in the storage unit 32 as identification information of an infected person.

The identification information distribution unit 314 transmits identification information of an infected person(s) stored in the storage unit 32 to the mobile terminal 10 at predetermined intervals. The predetermined interval is, for example, once a day.

The storage unit 32 includes a storage device such as an HDD, an SSD, or a flash memory. The storage unit 32 stores place information indicating the place where the display means 20 is installed. Further, the storage unit 32 stores identification information of an infected person(s). The storage unit 32 may store a plurality of types of identification information of infected persons.

The receiving unit 33 and the transmitting unit 34 connect to the network N, which may be the Internet, and performs wired or wireless communication. The receiving unit 33 receives information from the mobile terminal 10 and the display means 20 through the network N. The transmitting unit 34 transmits information to the mobile terminal 10 and the display means 20 through the network N.

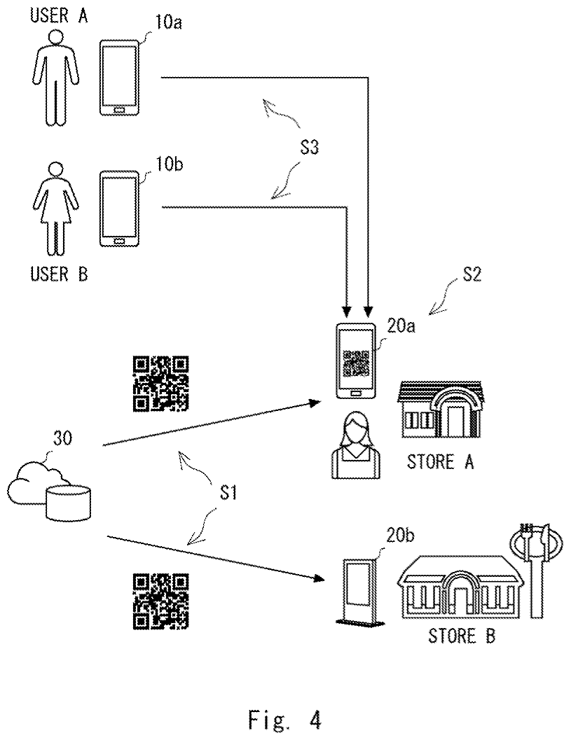

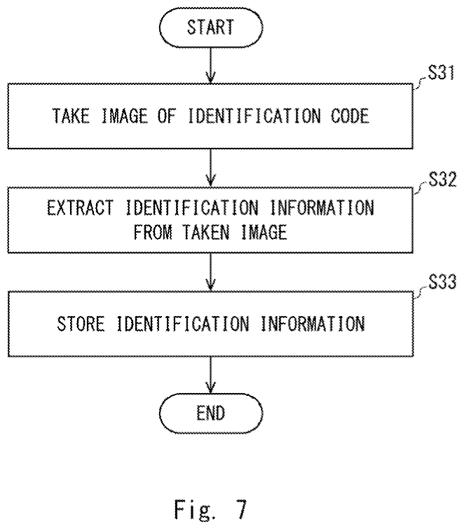

Next, operations performed by the proximity detection system 2 according to the second example embodiment will be described by using an example shown in FIGS. 4 to 11. The proximity detection system 2 performs an operation for acquiring a place shown in FIGS. 4 to 7, and an operation for identifying a proximity person (i.e., a person who had been in proximity to an infected person) and for a notification thereof shown in FIGS. 7 to 11.

In the example shown in FIGS. 4 to 11, a mobile terminal 10a, a mobile terminal 10b, and a mobile terminal 10c are mobile terminals 10 used by a user A, a user B, and a user C, respectively, and each of them has a configuration similar to that of the mobile terminal 10. Display means 20a and display means 20b are display means 20 installed in a store A and a store B, respectively, and each of them has a configuration similar to that of the display means 20.

Firstly, the operation for acquiring a place performed in the proximity detection system 2 according to the second example embodiment will be described with reference to FIGS. 4 to 11.

FIG. 4 is a schematic diagram showing an operation for acquiring a place performed in the proximity detection system 2 according to the second example embodiment. In the operation for acquiring a place, a history of behaviors of the user using the mobile terminal 10 is recorded.

Firstly, as shown in FIG. 4, the management server 30 creates an identification code and displays the created identification code on the display means 20a and 20b (Step S1).

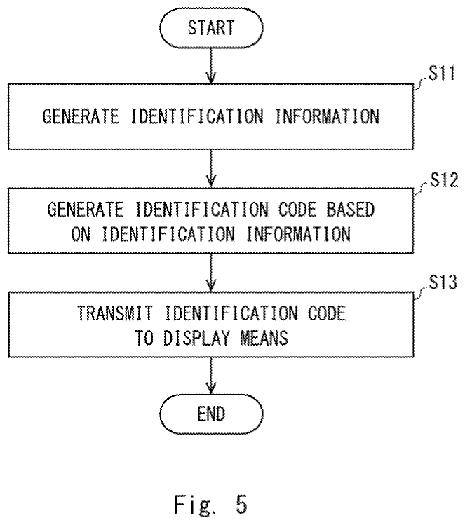

An operation for creating an identification code and displaying the created identification code on the display means 20a, performed in the management server according to the second example embodiment will be described hereinafter in detail with reference to FIG. 5. FIG. 5 is a flowchart showing an operation for creating an identification code and displaying the created identification code on the display means 20, performed in the management server 30 according to the second example embodiment.

As shown in FIG. 5, firstly, the identification information generation unit 311 of the management server 30 generates identification information (Step S11). The identification information is expressed by a string of numbers and/or a string of characters (e.g., alphabets). A combination of the place of the display means 20a and a time period is assigned to the identification information. Next, the identification code generation unit 312 generates an identification code based on the identification information (Step S12). For example, the identification code generation unit 312 generates an identification code by encoding the identification information. Next, the identification code generation unit 312 transmits the generated identification code to the display means 20a (Step S13).

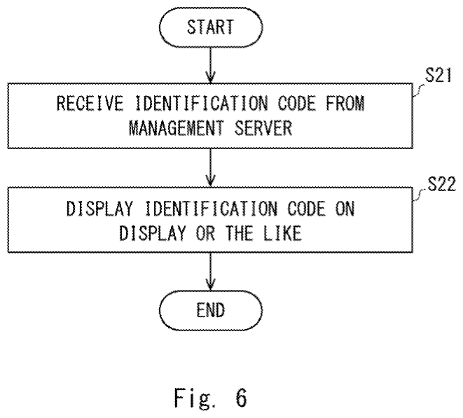

Next, as shown in FIG. 4, the display means 20a displays the identification code received from the management server 30 (Step S2). The operation for displaying the identification code performed in the display means 20a according to the second example embodiment will be described in detail with reference to FIG. 6. FIG. 6 is a flowchart showing the operation for displaying the identification code performed in the display means 20a according to the second example embodiment.

As shown in FIG. 6, firstly, the receiving unit 24 of the display means 20a receives an identification code from the management server 30 (Step S21). Next, the identification code output processing unit 211 of the control unit 21 displays the received identification code on a display or the like through the output unit 23 (Step S22).

Next, as shown in FIG. 4, the user A using the mobile terminal 10a visits the store A where the display means 20a is installed. The user B using the mobile terminal 10b also visits the store A in the same time period. Operations performed by the mobile terminal 10a will be mainly described hereinafter, and operations performed by the mobile terminal 10b are similar to those performed by the mobile terminal 10a.

After the user A visits the store A, the mobile terminal 10a stores an identification code displayed by the display means 20a according to an operation performed by the user A (Step S3). The operation for storing an identification code performed in the mobile terminal 10a according to the second example embodiment will be described hereinafter in detail with reference to FIG. 7. FIG. 7 is a flowchart showing the operation for storing an identification code performed in the mobile terminal 10 according to the second example embodiment.

As shown in FIG. 7, firstly, the image pickup unit 12 of the mobile terminal 10a takes an image of an identification code displayed by the display means 20a (Step S31). Then, the image pickup unit 12 supplies the taken image to the control unit 11. Next, the extraction unit 111 of the control unit 11 decodes the identification code contained in (i.e., shown in) the taken image and thereby extracts identification information from the identification code (Step S32). Next, the extraction unit 111 stores the extracted identification information in the storage unit 13 (Step S33). Note that even if the user had been in proximity to a person determined as an infected person in the past, if it is presumed that this person was not yet infected with the infectious disease at the time of the proximity, it is considered that there is no risk that the infectious disease is transmitted to the user due to the proximity. Therefore, the storage unit 13 may retain the identification information only for a predetermined period, for example, only for 14 days. The predetermined period is determined based on the incubation period or the like of the infectious disease.

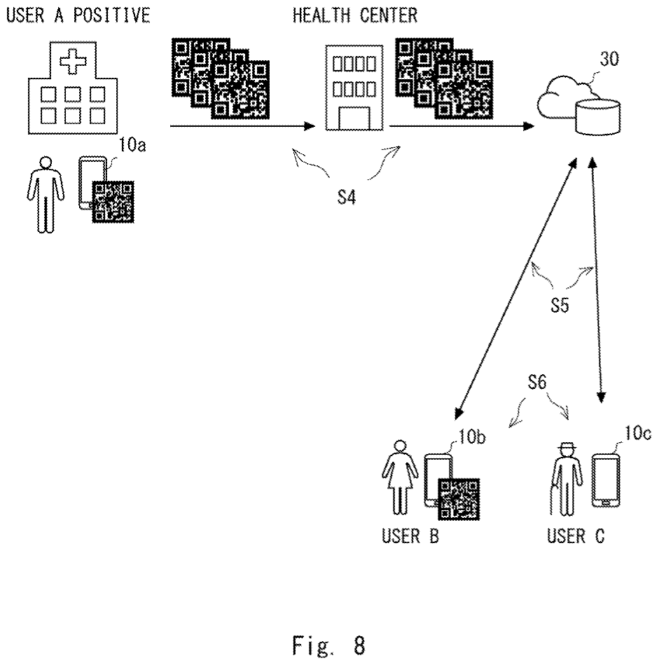

Next, an operation for identifying a proximity person (i.e., a person who had been in proximity to an infected person) and for a notification thereof, performed in the proximity detection system 2 according to the second example embodiment will be described with reference to FIGS. 8 to 11. FIG. 8 is a schematic diagram showing the operation for identifying a proximity person and for a notification thereof, performed in the proximity detection system 2 according to the second example embodiment.

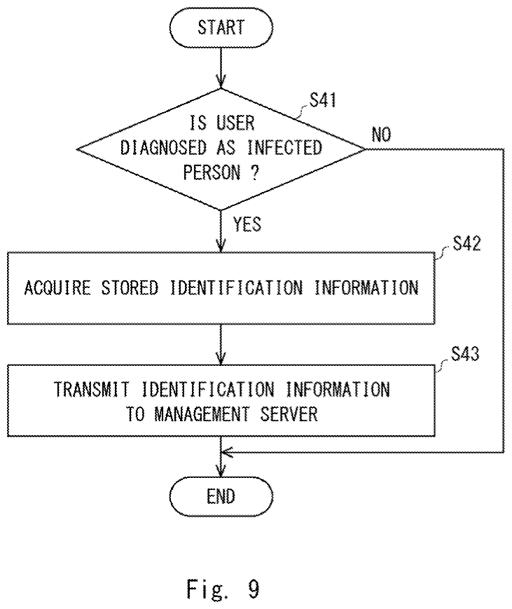

The user A is diagnosed as being infected by an infectious disease, i.e., diagnosed as an infected person at a hospital. When the user A is diagnosed as an infected person, the mobile terminal 10a of the user A transmits identification information stored in the mobile terminal 10a to the management server 30 (Step S4). FIG. 9 is a flowchart showing an operation for transmitting identification information to the management server 30 performed in the mobile terminal 10 according to the second example embodiment. Note that when the user transmits identification information from the mobile terminal 10a to the management server 30, the user is required to enter a password that is known only to specific persons such as a medical staff or the like. In this way, it is possible to prevent mobile terminals 10 of users who are not diagnosed as infected persons from accidentally transmitting identification information therefrom.

As shown in FIG. 9, firstly, when the user A is diagnosed as an infected person (Step S41 YES), the registration unit 114 acquires identification information from the storage unit 13 (Step S42). The identification information stored in the storage unit 13 contains identification information extracted from an identification code displayed by the display means 20a shown in FIG. 4. Next, the registration unit 114 transmits the acquired identification information to the management server 30 through the transmitting unit 15 (Step S43).

In the example shown in FIG. 9, the management server 30 is administered by a health center. Specifically, the health center may comprehensively determine, from the identification information received from the mobile terminal 10a of the infected person and other information about the infected person, whether or not to register the user A as an infected person in the management server 30. By doing so, it is possible to prevent incorrect information from being registered by, for example, an operation mistake made by the user A or by a mischievous or malicious operation.

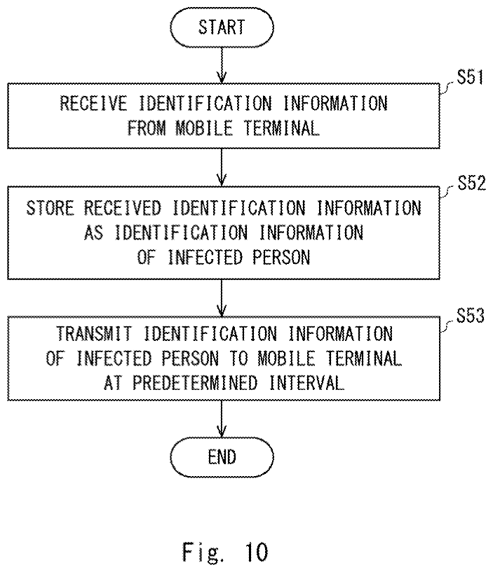

Next, as shown in FIG. 8, the management server 30 stores the identification information of the mobile terminal 10a as the identification information of the infected person, and transmits the identification information of the infected person to the mobile terminals 10b and 10c at predetermined intervals (Step S5). The predetermined interval is, for example, once a day, and at this interval, the management server 30 collectively transmits identification information of infected persons (e.g., a plurality of pieces of identification information of a plurality of infected persons) to the mobile terminals 10b and 10c. Operations for storing the identification information of the mobile terminal 10a as the identification information of the infected person and transmitting identification information of an infected person(s) to the mobile terminals 10b and 10c at predetermined intervals, performed in the management server 30 according to the second example embodiment will be described hereinafter in detail. FIG. 10 is a flowchart showing an operation for transmitting identification information of an infected person(s) to the mobile terminal 10 performed in the management server 30 according to the second example embodiment.

Firstly, as shown in FIG. 10, the receiving unit 33 of the management server 30 receives identification information from the mobile terminal 10a (Step S51). Next, the identification information registration unit 313 of the control unit 31 stores the received identification information of the mobile terminal 10a in the storage unit 32 as identification information of an infected person (Step S52).

Since the identification information of the infected person stored in the storage unit 32 is a string of numbers and a string of characters, it contains no information by which the place of the individual is specified and no information by which the individual is identified. Therefore, the personal information of the infected person is not be specified. Therefore, the privacy of the infected person can be protected.

Next, the identification information distribution unit 314 of the management server 30 transmits the identification information of the infected person stored in the storage unit 32 to the mobile terminal 10b through the transmitting unit 34 at predetermined intervals (Step S53). The transmitted identification information of the infected person contains the identification information of the mobile terminal 10a.

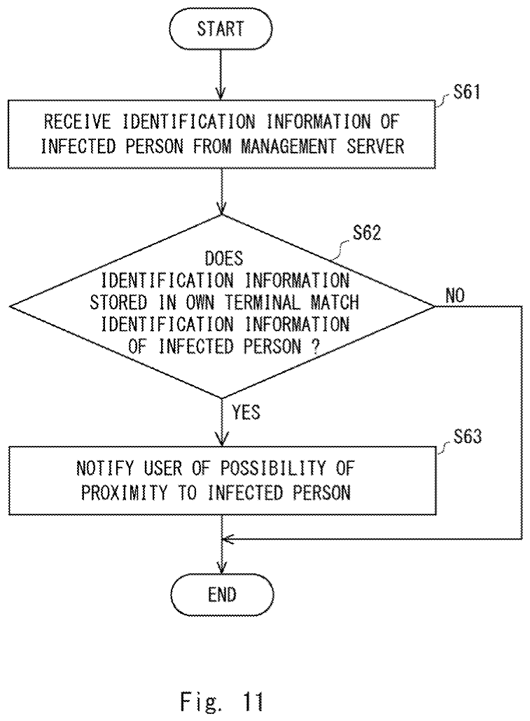

Next, as shown in FIG. 8, each of the mobile terminals 10b and 10c determines whether or not the identification information of the infected person received from the management server 30 matches the identification information stored in the user's own mobile terminal (i.e., the mobile terminal 10b or 10c itself), and notifies its user about the proximity to the infected person (Step S6). Operations for notifying the user B or C about the proximity to the infected person performed in each of the mobile terminals 10b and 10c according to the second example embodiment will be described hereinafter in detail with reference to FIG. 11. FIG. 11 is a flowchart showing an operation for notifying a user about proximity to an infected person performed in the mobile terminal 10 according to the second example embodiment.

Firstly, the operation for notifying the user B about proximity to an infected person performed by the mobile terminal 10b according to the second example embodiment will be described in detail with reference to FIG. 11.

As shown in FIG. 11, the acquisition unit 112 of the mobile terminal 10b receives identification information of an infected person from the management server 30 through the receiving unit 14 (Step S61). Next, the determination unit 113 determines whether or not the identification information stored in the storage unit 13 (identification information stored in the user's own mobile terminal) matches the identification information of the infected person acquired by the acquisition unit 112 (Step S62). When the identification information stored in the user's own mobile terminal matches the identification information of the infected person (Step S62 YES), the determination unit 113 notifies the user of the possibility of the proximity to the infected person through the output unit 16 (Step S63 YES). As shown in FIG. 4, since the user B using the mobile terminal 10b visited the store A in the time period in which the user A, who is the infected person, also visited the store A, the identification information stored in the mobile terminal 10b of the user B matches that stored in the mobile terminal 10a of the user A. Therefore, the determination unit 113 of the mobile terminal 10b notifies the user B of the possibility of the proximity to the infected person through the output unit 16.

Next, the operation for notifying the user C about proximity to an infected person performed by the mobile terminal 10c according to the second example embodiment will be described in detail with reference to FIG. 11.

As shown in FIG. 11, the acquisition unit 112 of the mobile terminal 10c receives identification information of an infected person from the management server 30 through the receiving unit 14 (Step S61). Next, the determination unit 113 determines whether or not the identification information stored in the storage unit 13 (identification information stored in the user's own mobile terminal) matches the identification information of the infected person acquired by the acquisition unit 112 (Step S62). When the identification information stored in the user's own mobile terminal does not match the identification information of the infected person (Step S62 NO), the mobile terminal 10c terminates the process. As shown in FIG. 4, since the user C using the mobile terminal 10c did not visit the store A in the time period in which the user A, who is the infected person, visited the store A, the identification information stored in the mobile terminal 10c of the user C does not match that stored in the mobile terminal 10a of the user A. Therefore, the determination unit 113 of the mobile terminal 10c does not notify the user C of the possibility of the proximity to the infected person through the output unit 16.

The proximity detection system 2 according to the second example embodiment can notify the user of the mobile terminal 10 of the possibility of the proximity to an infected person by determining whether or not the identification information stored in the user's own mobile terminal (i.e., the mobile terminal owned by the user) matches the identification information of the mobile terminal owned by the infected person.

Therefore, in the proximity detection system 2, even people who hesitate to use the system because of concern about their privacy or the like can use the system and grasp (i.e., know) the possibility of the proximity to an infected person. That is, in the proximity detection system 2, it is possible to easily grasp the possibility of the proximity to an infected person while the privacy is taken into consideration.

### <Example of Hardware Configuration>

Each functional component of the mobile terminal 10, the display means 20, and the management server 30 may be implemented by hardware (e.g., a hard-wired electronic circuit) that realizes the functional component, or by a combination of hardware and software (e.g., a combination of an electronic circuit and a program for controlling the electronic circuit). An example case where each functional component of the mobile terminal 10, the display means 20, and the management server 30 is implemented by a combination of hardware and software will be further described hereinafter.

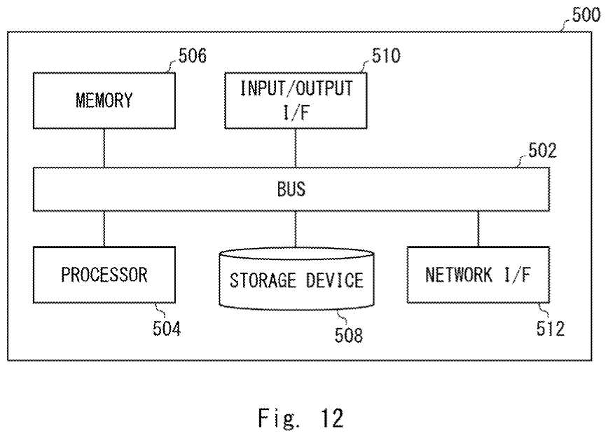

FIG. 12 is a block diagram showing a hardware configuration of a computer 500. Each of the mobile terminal 10, the display means, and the management server 30 can be implemented by the computer 500 having the hardware configuration shown in FIG. 12. The computer 500 that implements the mobile terminal 10 is a portable computer such as a smartphone or a tablet-type computer. Meanwhile, the computer 500 that implements the display means 20 or the management server 30 may be a portable computer or a stationary computer such as a PC (Personal Computer). The computer 500 may be a dedicated computer designed to implement the mobile terminal 10, the display means 20, or the management server 30, or may be a general-purpose computer. Further, the computer 500 may be a stationary computer such as a PC (Personal Computer).

For example, the computer 500 can have desired functions by installing a certain application(s) in the computer 500. For example, each function of the mobile terminal 10 is implemented in the computer 500 by installing an application for that function of the mobile terminal 10 in the computer 500. The same applies to the display means 20 and the management server 30.

The computer 500 includes a bus 502, a processor 504, a memory 506, a storage device 508, an input/output interface (I/F) 510, and a network interface (I/F) 512. The bus 502 is a data transmission path through which the processor 504, the memory 506, the storage device 508, the input/output interface 510, and the network interface 512 transmit and receive data to and from each other. However, the method for connecting the processor 504 and the like to each other is not limited to connections through buses.

The processor 504 is any of various processors such as a CPU (Central Processing Unit), a GPU (Graphics Processing Unit), or an FPGA (Field-Programmable Gate Array). The memory 506 is a main storage device implemented by using a RAM (Random Access Memory) or the like. The storage device 508 is an auxiliary storage device implemented by using a hard disk drive, an SSD (Solid State Drive), a memory card, or a ROM (Read Only Memory).

The input/output interface 510 is an interface for connecting the computer 500 with an input/output device(s). For example, an input device such as a keyboard and an output device such as a display device are connected to the input/output interface 510.

The network interface 512 is an interface for connecting the computer 500 to a network. The network may be a LAN (Local Area Network) or a WAN (Wide Area Network).

In the storage device 508, programs for implementing desired functions are stored. For example, a program(s) for implementing the functions of the mobile terminal 10 (a program(s) for the aforementioned application for implementing the functions of the mobile terminal 10) is stored in the storage device 508 included in the computer 500 for implementing the mobile terminal 10. The processor 504 implements each functional component of the mobile terminal 10, the display means 20, and the management server 30 by loading the program(s) into the memory 506 and executing the loaded program(s). The same applies to the display means 20 and the management server 30.

Note that the present invention is not limited to the above-described example embodiments, and they may be modified as appropriate without departing from the scope of the invention.

### Modified Example 1

In a modified example 1 of the proximity detection system 2 according to the second example embodiment, a user may be notified of proximity to an infected person by making a decision on the identification code instead of on the identification information.

Specifically, the mobile terminal 10 stores an identification code displayed by the display means 20 in the storage unit 13. Next, when the user of the mobile terminal 10 is diagnosed as an infected person, the mobile terminal 10 transmits the identification code to the management server 30. Then, the management server 30 stores the received identification code in the storage unit 32 as an identification code of an infected person. Next, the management server transmits identification code of an infected person(s) to the mobile terminal 10 at predetermined intervals. Next, when mobile terminal 10 acquires the identification code of the infected person from the management server 30, it determines whether or not the identification code stored in the storage unit 13 of the user's own mobile terminal matches the identification code of the infected person. The mobile terminal 10 notifies the user of information about proximity of the user to the infected person based on the result of the determination.

### Modified Example 2

In a modified example 2 of the proximity detection system 2 according to the second example embodiment, the management server 30 may generate an identification code, or the display means 20 may generate an identification code by a method similar to that by which the management server 30 generates an identification code, and make (i.e., instruct) the output unit 23 display the generated identification code.

Specifically, the display means 20 further includes an identification information generation unit 311 and an identification code generation unit 312. The identification information generation unit 311 generates identification information. The identification code generation unit 312 generates an identification code based on the generated identification information. Then, the identification code generation unit 312 displays the generated identification code on a display or the like through the output unit 23.

The program may be stored in various types of non-transitory computer readable media and thereby supplied to the computer. The non-transitory computer readable media includes various types of tangible storage media. Examples of non-transitory computer readable media include magnetic recording media (e.g., flexible disks, magnetic tapes, hard disk drives), magneto-optical recording media (e.g., magneto-optical disks), CD-ROM (Read Only Memory), CD-R, CD-R/W, and semiconductor memories (e.g., mask ROM, PROM (Programmable ROM), EPROM (Erasable PROM), flash ROM, and RAM (random access memory)). Further, the programs may be supplied to the computer by using various types of transitory computer readable media. Examples of the transitory computer readable media include an electrical signal, an optical signal, and an electromagnetic wave. The transitory computer readable media can be used to supply a program to a computer through a wired communication line (e.g., an electric wire and an optical fiber) or a radio communication line.

The whole or part of the example embodiments disclosed above can be described as, but not limited to, the following supplementary notes.

### (Supplementary Note 1)

A non-transitory computer readable medium storing a program for causing a computer to:
extract, from an image of an identification code generated based on identification information assigned to a place and displayed at the place, the identification information, and store the extracted identification information in a storage device;
acquire identification information stored in a storage device of a mobile terminal of an infected person;
determine whether or not the acquired identification information matches the identification information stored in the storage device of a user's own mobile terminal; and
notify the user of information about proximity of the user to the infected person based on a result of the determination.

### (Supplementary Note 2)

The non-transitory computer readable medium storing the program for the computer described in Supplementary note 1, wherein the identification information is assigned to each combination of a place and a time.

### (Supplementary Note 3)

The non-transitory computer readable medium storing the program for the computer described in Supplementary note 1 or 2, wherein the notifying of the user of the information about the proximity to the infected person based on the result of the determination includes notifying, when the acquired identification information matches the identification information stored in the storage device of the user's own mobile terminal, the user that there is a possibility of the proximity to the infected person.

### (Supplementary Note 4)

The non-transitory computer readable medium storing the program for the computer described in any one of Supplementary notes 1 to 3, wherein the acquiring of the identification information stored in the storage device of the mobile terminal of the infected person includes acquiring the identification information stored in the storage device of the mobile terminal of the infected person at predetermined intervals.

### (Supplementary Note 5)

A mobile terminal comprising:
an extraction unit configured to extract, from an image of an identification code generated based on identification information assigned to a place and displayed at the place, the identification information, and store the extracted identification information in a storage device;
an acquisition unit configured to acquire identification information stored in a storage device of a mobile terminal of an infected person; and
a determination unit configured to determine whether or not the acquired identification information matches the identification information stored in the storage device of a user's own mobile terminal, wherein
the determination unit notifies the user of information about proximity of the user to the infected person based on a result of the determination.

### (Supplementary Note 6)

The mobile terminal described in Supplementary note 5, wherein the identification information is assigned to each combination of a place and a time.

### (Supplementary Note 7)

The mobile terminal described in Supplementary note 5 or 6, wherein the determination unit notifies, when the acquired identification information matches the identification information stored in the storage device of the user's own mobile terminal, the user that there is a possibility of the proximity to the infected person.

### (Supplementary Note 8)

The mobile terminal described in any one of Supplementary notes 5 to 7, wherein the acquisition unit acquires the identification information stored in the storage device of the mobile terminal of the infected person at predetermined intervals.

### (Supplementary Note 9)

A proximity detection system comprises:
the mobile terminal described in any one of Supplementary notes 5 to 8; and
display means, wherein
the display means generates an identification code corresponding to a place where the display means is installed, and displays the generated identification code.

### (Supplementary Note 10)

The proximity detection system described in Supplementary note 9, wherein the display means generates the identification code corresponding to a combination of a time and the place where the display means is installed at predetermined intervals.

### (Supplementary Note 11)

The proximity detection system described in Supplementary note 9 or 10, wherein the display means generates the identification code by generating a random number, generating a partial image corresponding to the random number, and disposing the partial image at a predetermined position.

### (Supplementary Note 12)

A method comprising:
extracting, by a mobile terminal, from an image of an identification code generated based on identification information assigned to a place and displayed at the place, the identification information, and storing the extracted identification information in a storage device;
acquiring, by the mobile terminal, identification information stored in a storage device of a mobile terminal of an infected person;
determining, by the mobile terminal, whether or not the acquired identification information matches the identification information stored in the storage device of a user's own mobile terminal; and
notifying, by the mobile terminal, the user of information about proximity of the user to the infected person based on a result of the determination.

### (Supplementary Note 13)

The method described in Supplementary note 12, wherein the identification information is assigned to each combination of a place and a time.

### (Supplementary Note 14)

The method described in Supplementary note 12 or 13, wherein the notifying of the user of the information about the proximity to the infected person based on the result of the determination includes notifying, when the acquired identification information matches the identification information stored in the storage device of the user's own mobile terminal, the user that there is a possibility of the proximity to the infected person.

### (Supplementary Note 15)

The method described in any one of Supplementary notes 12 to 14, wherein the acquiring of the identification information stored in the storage device of the mobile terminal of the infected person includes acquiring the identification information stored in the storage device of the mobile terminal of the infected person at predetermined intervals.

### REFERENCE SIGNS LIST

2 PROXIMITY DETECTION SYSTEM
10 MOBILE TERMINAL
10a MOBILE TERMINAL
10b MOBILE TERMINAL
10c MOBILE TERMINAL
11 CONTROL UNIT
12 IMAGE PICKUP UNIT
13 STORAGE UNIT
14 RECEIVING UNIT
15 TRANSMITTING UNIT
16 OUTPUT UNIT
20 DISPLAY MEANS
20a DISPLAY MEANS
20b DISPLAY MEANS
21 CONTROL UNIT
22 STORAGE UNIT
23 OUTPUT UNIT
24 RECEIVING UNIT
25 MANAGEMENT SERVER
31 CONTROL UNIT
32 STORAGE UNIT
33 RECEIVING UNIT
34 TRANSMITTING UNIT
111 EXTRACTION UNIT
112 ACQUISITION UNIT
113 DETERMINATION UNIT
114 REGISTRATION UNIT
211 IDENTIFICATION CODE OUTPUT PROCESSING UNIT
311 IDENTIFICATION INFORMATION GENERATION UNIT
312 IDENTIFICATION CODE GENERATION UNIT
313 IDENTIFICATION INFORMATION REGISTRATION UNIT
314 IDENTIFICATION INFORMATION DISTRIBUTION UNIT
500 COMPUTER
502 BUS
504 PROCESSOR
506 MEMORY
508 STORAGE DEVICE
510 INPUT/OUTPUT I/F
512 NETWORK I/F
N NETWORK

What is claimed is:

1. A non-transitory computer readable medium storing a program for causing a computer to:
extract, from an image of an identification code generated based on identification information assigned to a place and displayed at the place, the identification information, and store the extracted identification information in a storage device;
acquire identification information stored in a storage device of a mobile terminal of an infected person;
determine whether or not the acquired identification information matches the identification information stored in the storage device of a user's own mobile terminal; and
notify the user of information about proximity of the user to the infected person based on a result of the determination.

2. The non-transitory computer readable medium storing the program for the computer according to claim 1, wherein the identification information is assigned to each combination of a place and a time.

3. The non-transitory computer readable medium storing the program for the computer according to claim 1, wherein the notifying of the user of the information about the proximity to the infected person based on the result of the determination includes notifying, when the acquired identification information matches the identification information stored in the storage device of the user's own mobile terminal, the user that there is a possibility of the proximity to the infected person.

4. The non-transitory computer readable medium storing the program for the computer according to claim 1, wherein the acquiring of the identification information stored in the storage device of the mobile terminal of the infected person includes acquiring the identification information stored in the storage device of the mobile terminal of the infected person at predetermined intervals.

5. A mobile terminal comprising:
at least one memory storing instructions; and
at least one processor that is configured to execute the instructions to:
extract, from an image of an identification code generated based on identification information assigned to a place and displayed at the place, the identification information, and store the extracted identification information in a storage device;
acquire identification information stored in a storage device of a mobile terminal of an infected person; and
determine whether or not the acquired identification information matches the identification information stored in the storage device of a user's own mobile terminal, wherein
notify the user of information about proximity of the user to the infected person based on a result of the determination.

6. The mobile terminal according to claim 5, wherein the identification information is assigned to each combination of a place and a time.

7. The mobile terminal according to claim 5, wherein the at least one processor is configured to further to notify, when the acquired identification information matches the identification information stored in the storage device of the user's own mobile terminal, the user that there is a possibility of the proximity to the infected person.

8. The mobile terminal according to claim 5, wherein the at least one processor is configured to further to acquire the identification information stored in the storage device of the mobile terminal of the infected person at predetermined intervals.

9.-11. (canceled)

12. A method comprising:
extracting, by a mobile terminal, from an image of an identification code generated based on identification information assigned to a place and displayed at the place, the identification information, and storing the extracted identification information in a storage device;
acquiring, by the mobile terminal, identification information stored in a storage device of a mobile terminal of an infected person;
determining, by the mobile terminal, whether or not the acquired identification information matches the identification information stored in the storage device of a user's own mobile terminal; and
notifying, by the mobile terminal, the user of information about proximity of the user to the infected person based on a result of the determination.

13. The method according to claim 12, wherein the identification information is assigned to each combination of a place and a time.

14. The method according to claim 12, wherein the notifying of the user of the information about the proximity to the infected person based on the result of the determination includes notifying, when the acquired identification information matches the identification information stored in the storage device of the user's own mobile terminal, the user that there is a possibility of the proximity to the infected person.

15. The method according to claim 12, wherein the acquiring of the identification information stored in the storage device of the mobile terminal of the infected person includes acquiring the identification information stored in the storage device of the mobile terminal of the infected person at predetermined intervals.
